(19) United States
(12) Patent Application Publication
(10) Pub. No.: US 2019/0139623 A1
(43) Pub. Date: May 9, 2019

(54) DISPLAY OF ESTIMATED PARENTAL CONTRIBUTION TO ANCESTRY

(71) Applicant: 23andMe, Inc., Mountain View, CA (US)

(72) Inventors: Katarzyna Bryc, Redwood City, CA (US); Eric Yves Jean-Marc Durand, Sceaux (FR); Joanna Louise Mountain, Menlo Park, CA (US); Robin Patrick Smith, Mountain View, CA (US); Peilun Shan, Redmond, WA (US); Bradley Kittredge, San Francisco, CA (US)

(21) Appl. No.: 16/240,641

(22) Filed: Jan. 4, 2019

Related U.S. Application Data
(63) Continuation of application No. 14/924,552, filed on Oct. 27, 2015.
(60) Provisional application No. 62/072,275, filed on Oct. 29, 2014.

(51) Int. Cl. G16B 10/00

(52) U.S. Cl. CPC G16B 10/00

(57) ABSTRACT

Estimating parental contribution of ancestry includes: obtaining a set of ancestry assignment data associated with an individual's genotype data, at least some of the ancestry assignment data indicating that one or more segments of the individual's genotype data is deemed to be associated with a specific ancestry; determining whether in the individual's genotype data there is at least one confirmed region of overlapping ancestry assignment associated with the specific ancestry; in the event that it is determined that there is at least one confirmed region of overlapping ancestry assignment associated with the specific ancestry: specifying that parental contribution of the specific ancestry is made by both parents of the individual; in the event that it is determined that there is no confirmed region of overlapping ancestry assignment associated with the specific ancestry: statistically determining whether the parental contribution to the specific ancestry is made by only one parent of the individual or by both parents of the individual, the determination being based at least in part on one or more lengths of the one or more segments deemed to be associated with the specific ancestry; and outputting information pertaining to the parental contribution to the specific ancestry.

### CROSS REFERENCE TO OTHER APPLICATIONS

This application is a continuation of U.S. patent application Ser. No. 14/924,552 entitled DISPLAY OF ESTIMATED PARENTAL CONTRIBUTION TO ANCESTRY filed on Oct. 27, 2015, which claims priority to U.S. Provisional Patent Application No. 62/072,275 entitled ESTIMATION OF PARENTAL CONTRIBUTION TO ANCESTRY filed Oct. 29, 2014; all of the above prior applications are incorporated by reference in their entireties for all purposes.

### BACKGROUND OF THE INVENTION

Genealogy is the study of family lineage and history. Traditional genealogical studies typically rely on surnames and historical records (e.g., registries of births and marriages, etc.) to determine a person's ancestry. However, such techniques are very limited since many ancestry determinations are difficult to make merely based on records. For example, for an individual with mixed ancestries, it is often difficult to know which specific ancestries the individual has, or whether one parent or both contributed to those ancestries since it is rare that a person would have a complete record of family history.

In recent years, techniques have been developed using individuals' genetic information to trace ancestries. Humans have 23 pairs of chromosomes, of which 22 are autosomal chromosomes. During meiosis, a maternal chromosome and a paternal chromosome pair up to form a pair of homologous chromosomes. Homologous autosomal chromosomes have the same genes in the same locations, but can have different variants of the same genes. Following the homologous chromosomes pairing, genetic recombination occurs. As a result, the offspring has genotypes that are new and different combinations of the parental alleles.

Certain existing genetics-based ancestry assignment techniques can be used to determine, for an autosomal chromosome, the geological ancestries of various segments on the chromosome. For example, certain existing ancestry assignment tools compare the genotype data of an individual with various reference models known to correspond to certain geographical ancestries of origin (e.g., Native American, Northern European, Asian, etc.) to determine the individual's ancestries. Existing techniques, however, have certain limitations and present various technical challenges. For instance, without additional genotype data from the individual's parents, existing techniques usually cannot determine whether a particular ancestry is attributed to one parent only or to both parents. Moreover, the genotype information (e.g., DNA sequence information or marker information) obtained using genotyping chips is typically unphased and subject to a phasing process. The phasing process can introduce errors, making it more difficult to correctly determine the parental contribution of ancestry. Additionally, the ancestry assignment tools sometimes can also introduce errors when making the ancestry assignments, making the assignment results inaccurate.

### BRIEF DESCRIPTION OF THE DRAWINGS

Various embodiments of the invention are disclosed in the following detailed description and the accompanying drawings.

FIG. 1 is a functional diagram illustrating a programmed computer system for estimating parental contribution of ancestry in accordance with some embodiments.

FIG. 2 is a block diagram illustrating an embodiment of a system for estimating parental contributions of ancestry.

FIG. 3 is a diagram illustrating an example of a pair of homologous chromosomes with an overlapping region of ancestry assignment.

FIG. 4 is a flowchart illustrating an embodiment of a process for displaying estimated parental contribution to ancestry.

FIG. 5 is a flowchart illustrating an embodiment of a process for determining whether there is at least one confirmed region of overlapping ancestry assignment associated with a specific ancestry.

FIG. 6 is a flowchart illustrating an embodiment of a process for determining the overlap threshold.

FIG. 7 is a flowchart illustrating an embodiment of a process to statistically determine whether the parental contribution to the specific ancestry is deemed to be made by one parent or by both parents of the individual.

FIG. 8 is a flowchart illustrating an embodiment of a process for determining the probability threshold.

FIG. 9 is an embodiment of a user interface screen illustrating the estimated parental ancestry contributions.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

How to estimate parental contribution to ancestry is described. In some embodiments, an individual's genotype data is used to estimate parental contribution to a specific ancestry. As used herein, parental contribution to an ancestry refers to whether one parent or both parents contributed to a specific ancestry found to correspond to certain parts of the individual's genome. The technique includes identifying whether there is a confirmed region of overlapping ancestry assignment based on the individual's genotype data, and in the event that there is no confirmed region of overlapping ancestry assignment, statistically determining parental contribution to the specific ancestry based at least in part on the chromosome segment lengths associated with the specific ancestry.

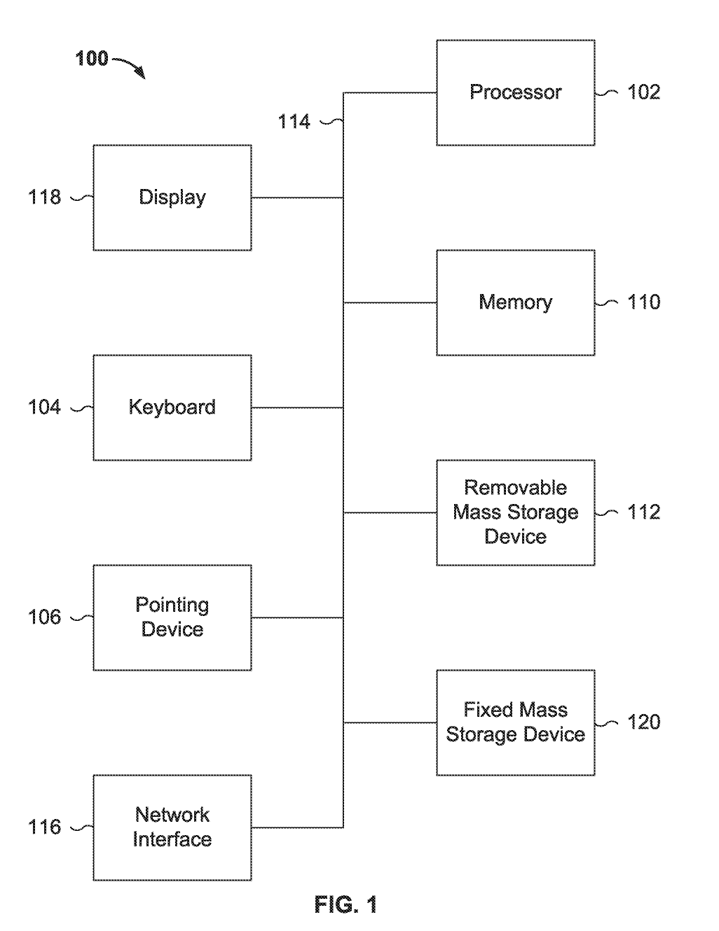

FIG. 1 is a functional diagram illustrating a programmed computer system for estimating parental contribution of ancestry in accordance with some embodiments. As will be apparent, other computer system architectures and configurations can be used to perform the estimation of parental contribution. Computer system 100, which includes various subsystems as described below, includes at least one microprocessor subsystem (also referred to as a processor or a central processing unit (CPU)) 102. For example, processor 102 can be implemented by a single-chip processor or by multiple processors. In some embodiments, processor 102 is a general purpose digital processor that controls the operation of the computer system 100. Using instructions retrieved from memory 110, the processor 102 controls the reception and manipulation of input data, and the output and display of data on output devices (e.g., display 118). In some embodiments, processor 102 includes and/or is used to provide elements 206-212 and/or executes/performs the processes described below.

Processor 102 is coupled bi-directionally with memory 110, which can include a first primary storage, typically a random access memory (RAM), and a second primary storage area, typically a read-only memory (ROM). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 102. Also as is well known in the art, primary storage typically includes basic operating instructions, program code, data and objects used by the processor 102 to perform its functions (e.g., programmed instructions). For example, memory 110 can include any suitable computer-readable storage media, described below, depending on whether, for example, data access needs to be bi-directional or uni-directional. For example, processor 102 can also directly and very rapidly retrieve and store frequently needed data in a cache memory (not shown).

A removable mass storage device 112 provides additional data storage capacity for the computer system 100, and is coupled either bi-directionally (read/write) or uni-directionally (read only) to processor 102. For example, storage 112 can also include computer-readable media such as magnetic tape, flash memory, PC-CARDS, portable mass storage devices, holographic storage devices, and other storage devices. A fixed mass storage 120 can also, for example, provide additional data storage capacity. The most common example of mass storage 120 is a hard disk drive. Mass storages 112, 120 generally store additional programming instructions, data, and the like that typically are not in active use by the processor 102. It will be appreciated that the information retained within mass storages 112 and 120 can be incorporated, if needed, in standard fashion as part of memory 110 (e.g., RAM) as virtual memory.

In addition to providing processor 102 access to storage subsystems, bus 114 can also be used to provide access to other subsystems and devices. As shown, these can include a display monitor 118, a network interface 116, a keyboard 104, and a pointing device 106, as well as an auxiliary input/output device interface, a sound card, speakers, and other subsystems as needed. For example, the pointing device 106 can be a mouse, stylus, track ball, or tablet, and is useful for interacting with a graphical user interface.

The network interface 116 allows processor 102 to be coupled to another computer, computer network, or telecommunications network using a network connection as shown. For example, through the network interface 116, the processor 102 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 102 can be used to connect the computer system 100 to an external network and transfer data according to standard protocols. For example, various process embodiments disclosed herein can be executed on processor 102, or can be performed across a network such as the Internet, intranet networks, or local area networks, in conjunction with a remote processor that shares a portion of the processing. Additional mass storage devices (not shown) can also be connected to processor 102 through network interface 116.

An auxiliary I/O device interface (not shown) can be used in conjunction with computer system 100. The auxiliary I/O device interface can include general and customized interfaces that allow the processor 102 to send and, more typically, receive data from other devices such as microphones, touch-sensitive displays, transducer card readers, tape readers, voice or handwriting recognizers, biometrics readers, cameras, portable mass storage devices, and other computers.

In addition, various embodiments disclosed herein further relate to computer storage products with a computer readable medium that includes program code for performing various computer-implemented operations. The computer-readable medium is any data storage device that can store data which can thereafter be read by a computer system. Examples of computer-readable media include, but are not limited to, all the media mentioned above: magnetic media such as hard disks, floppy disks, and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code, as produced, for example, by a compiler, or files containing higher level code (e.g., script) that can be executed using an interpreter.

The computer system shown in FIG. 1 is but an example of a computer system suitable for use with the various embodiments disclosed herein. Other computer systems suitable for such use can include additional or fewer subsystems. In addition, bus 114 is illustrative of any interconnection scheme serving to link the subsystems. Other computer architectures having different configurations of subsystems can also be utilized.

An individual's parent can have a portion of his or her genome that is deemed to be associated with a specific ancestry (e.g., a specific geographical region). If an individual inherits that portion of the parent's genome, the parent is said to have made a parental contribution of the specific ancestry with respect to the individual. In practice, the individual's parents' genome data is often unavailable; therefore, whether one parent or both parents have portions of their genomes associated with the specific ancestry is also unknown. The technique described below makes an estimation based on the individual's own genome data without requiring the individual's parents' genome data.

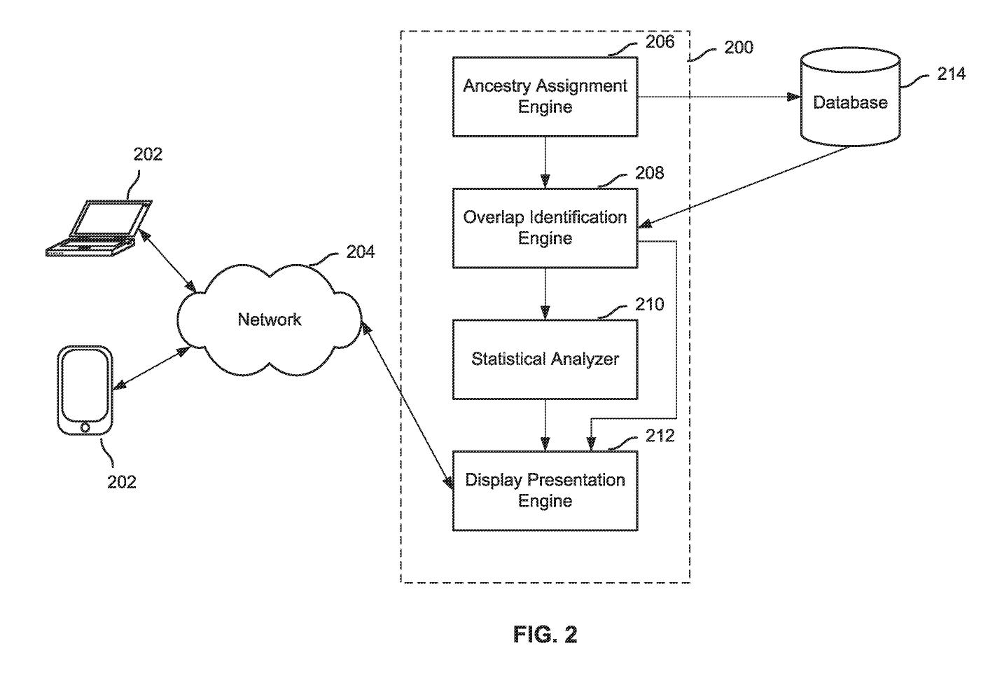

FIG. 2 is a block diagram illustrating an embodiment of a system for estimating parental contributions of ancestry.

In this example, a user uses a client device 202 to communicate with an ancestry estimation system 200 via a network 204. Examples of device 202 include a laptop computer, a desktop computer, a smart phone, a mobile device, a tablet device, a wearable networking device, or any other appropriate computing device.

Ancestry estimation system 200 is configured to estimate parental contributions of an individual's ancestry and present the estimation results for display. Ancestry estimation system 200 can be implemented on a networked platform (e.g., a server or cloud-based platform, a peer-to-peer platform, etc.) that supports various applications, such as 23andMe®′s personal genome service platform. For example, embodiments of the platform provide users with access (e.g., via appropriate user interfaces and communication channels implemented using browser-based applications, standalone applications, etc.) to their personal genetic information (e.g., genetic sequence information and/or genotype information obtained by assaying genetic materials such as blood or saliva samples). In some embodiments, the platform also allows users to connect with each other and share information.

In some embodiments, genetic samples (e.g., saliva, blood, etc.) are collected from individuals and analyzed using DNA microarray or other appropriate techniques. The individuals' genotype information is obtained (e.g., from genotyping chips directly or from genotyping services that provide assayed results) and stored in database 214. The genotype data can include fully sequenced genome data, Single Nucleotide Polymorphism (SNP) data, exonic data pertaining to exons (the coding portion of genes that are expressed), other assayed DNA marker data (e.g., short tandem repeats (STRs), Copy-Number Variants (CNVs), etc.), as well as any other appropriate form of genetic data pertaining to the individual's genome. In this example, the genotype data is used by system 200 to estimate parental contributions to individuals' ancestries. Results of the estimation can be stored to database 214 or any other appropriate storage unit. Although SNP-based DNA information is discussed for purposes of illustration, the technique is also applicable to other forms of genotype data.

In this example, system 200 includes an ancestry assignment engine 206, an overlap identification engine 208, a statistical analyzer engine 210, and a display presentation engine 212. In some embodiments, ancestry assignment engine 206 is implemented using an ancestry composition tool such as 23andMe's Geographic Ancestry Analyzer®, which determines ancestry composition based on the individual's genomic information and generates the ancestry assignment (also referred to as ancestry composition). Individuals with ancestries from different geographical regions are found to have different genetic variations in certain gene locations. In some embodiments, genome reference models are obtained based on genomes of reference individuals that are known to have specific ancestries. For example, a genome reference model can be obtained based on an un-admixed individual who is known to have four grandparents born in the same geographical region. For example, the Geographic Ancestry Analyzer® employs reference models from geographical regions such as Native America, Norther Europe, Southern Europe, and many other geographical regions or subregions. In some embodiments, segments of an individual's chromosomes are compared with the reference models to find matches and determine the most likely ancestry for each segment accordingly (e.g., if a particular chromosome segment is found to match a corresponding chromosome segment at the same location in the Scandinavian model, then that chromosome segment of the individual user is assigned Scandinavian ancestry). How to find chromosome segment matches and assigning ancestries is known to those skilled in the art. The ancestry assignment data can be stored in database 214, output to overlap identification engine 208 for further processing, or both.

Existing ancestry composition tools may produce results that contain errors, such as phasing errors or assignment errors. Phasing errors occur because many existing genotyping techniques read chromosome pairs and generate unphased readings that do not specify which one of the chromosome pairs a particular reading corresponds to. Unphased data needs to be phased to be useful for ancestry estimation purposes. Various phasing techniques that do not require parents' genotype data are used for phasing. Examples of such phasing techniques include the BEAGLE Genetic Analysis Software Package© developed by Brian Browning and the Finch technique developed by 23andMe®. These phasing techniques typically determine how the chromosome readings should be phased statistically (e.g., based on how frequently a particular combination of adjacent markers on a chromosome occurs in a particular population), which can lead to phasing errors. For example, suppose a portion of a chromosome pair has genotypes of A-T-C inherited from the one parent and G-T-A inherited from the other parent; the genotyping chip produces unphased readings of G/A-T/T-C/A, and the phasing technique can produce an incorrect phasing result indicating that genotypes of G-T-C are inherited from one parent and A-T-A from another. Although statistical techniques such as determining which combinations of genotypes frequently occur on the same chromosome can be used to improve results, phasing errors can still occur. Assignment errors are another type of common errors. They occur because the reference models do not include every single genotype but instead rely on markers to identify segments attributed to be specific ancestries, and some statistical uncertainty is expected around these inferences. Thus, the end of an individual's chromosome segment may match more than one reference model, and the final ancestry assignment of the end section can be different from the actual underlying ancestries corresponding to these sections, thus an assignment error can arise. For example, suppose that a first segment of an individual's chromosome matches the French reference model, and a second, adjacent chromosome segment matches the Italian model. Further, suppose that the end of the first segment matches the chromosomes at this location in both the French and the Italian reference models. Thus, this end segment can be associated with either French or Italian ancestry, even though the assignment indicates that it is French.

To determine parental contributions of ancestry, overlap identification engine 208 obtains ancestry assignment data directly from ancestry assignment engine 206 or from database 214. At least some of the obtained ancestry assignment data indicates that certain segments of an individual's genotype data are deemed to be associated with a specific ancestry. Overlap identification engine 208 identifies regions of overlapping ancestry assignment that correspond to the specific ancestry, and confirms whether such regions are actually overlapping regions or erroneously determined overlapping regions due to errors (e.g., phasing errors, assignment errors, etc.) present in the ancestry assignment data. A confirmed region of overlapping ancestry assignment indicates that both parents have contributed to the specific ancestry. The lack of any confirmed region of overlapping ancestry assignment leads to multiple possibilities, and further processing is performed by a statistical analyzer engine 210. In some embodiments, statistical analyzer engine 210 statistically determines whether the specific ancestry is deemed to be attributed to one parent only, or to both parents. As will be described in greater detail below, the determination is based at least in part on the length of the segments of the individual's genotype data deemed to be associated with the specific ancestry.

The engines described above can be implemented as software components executing on one or more processors, as hardware such as programmable logic devices and/or Application Specific Integrated Circuits designed to perform certain functions or a combination thereof. In some embodiments, the engines can be embodied by a form of software products which can be stored in a nonvolatile storage medium (such as optical disk, flash storage device, mobile hard disk, etc.), including a number of instructions for making a computer device (such as personal computers, servers, network equipment, etc.) implement the methods described in the embodiments of the present application. The engines may be implemented on a single device or distributed across multiple devices, including one or more devices similar to 100 of FIG. 1. The functions of the engines may be merged into one another or further split into multiple sub-components.

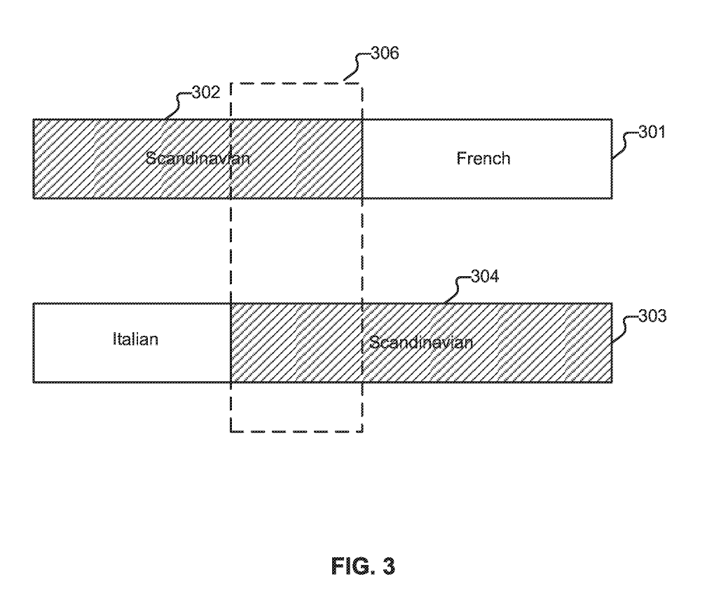

FIG. 3 is a diagram illustrating an example of a pair of homologous chromosomes with an overlapping region of ancestry assignment. In this example, a portion of a chromosome pair (chromosomes 301 and 303) is shown. In particular, chromosome segments 302 and 304 are both determined to be of Scandinavian ancestry based on known ancestry assignment techniques. Region 306 is referred to as a region of overlapping ancestry assignment, which can be identified by comparing the ancestry assignments of the chromosome pair. One possibility for the overlap is that both parents' genomes have Scandinavian ancestry in this region. Another possibility for the overlap is that there is an error made by the ancestry assignment engine, such as phasing errors or assignment errors, which has led to an incorrect overlapping assignment. The parental ancestry contribution estimation technique described below accounts for both possibilities. An overlapping region that is deemed to be likely due to both parents' genomes having ancestry in this region is referred to as a confirmed region of overlapping ancestry assignment.

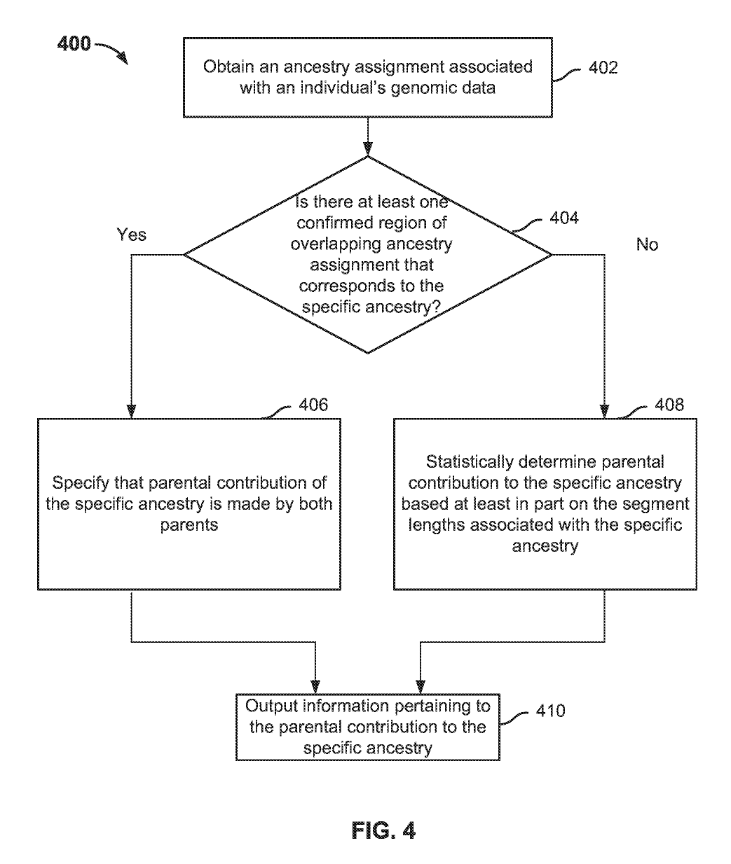

FIG. 4 is a flowchart illustrating an embodiment of a process for displaying estimated parental contribution to ancestry. Process 400 can be performed by a system such as 200.

At 402, a set of ancestry assignments associated with an individual's genotype data is obtained. It is assumed that the genotype data of the individual is originally unphased, then phased using a statistical phasing technique as discussed above, without requiring the individual's parents' genotype data. In some embodiments, an ancestry composition tool such as 23andMe's Geographic Ancestry Analyzer® is used to determine the ancestry composition of an individual's genome. As described above, in some embodiments, a given segment is assigned a most likely ancestry based on matching with reference models, and the assignments are stored in a database and/or output to the overlap identification engine. Any other appropriate techniques for assigning estimated ancestries to segments of the individual's genome can be used. In some embodiments, the ancestry assignments include specifications of the starting and ending positions of the segments and their assignments (e.g., chromosome 1, position 1-position 15, Scandinavian; chromosome 1, position 16-position 20, German, etc.). Other data formats can be used. For example, the chromosome identifiers and ancestries can be encoded to reduce memory use (e.g., 1:1-15:S, 1:16-20:G, etc.). In this case, the assignments associated with a specific ancestry (e.g., Scandinavian) are selected for further processing.

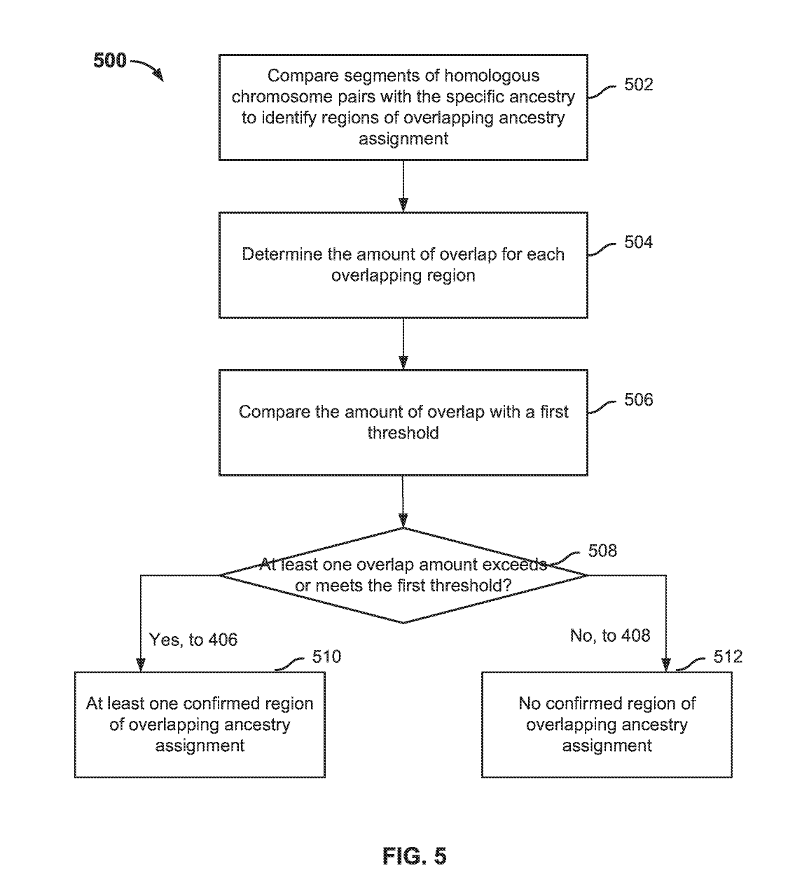

At 404, it is determined whether in the individual's genotype data there is at least one confirmed region of overlapping ancestry assignment that corresponds to the specific ancestry. A confirmed region of overlapping ancestry assignment refers to a region on a chromosome pair that is deemed to have an actual overlapping ancestry assignment due to both parents contributing to the same ancestry, rather than an incorrect overlapping assignment due to an error in the ancestry assignment technique. In some embodiments, the determination of whether an overlapping region is a confirmed region is based at least in part on the amount of overlap in an overlapping region to be confirmed. Details of the determination are described below in connection with FIG. 5.

If there is at least one confirmed region of overlapping ancestry assignment, at 406, it is specified that both parents have contributed to this ancestry. In some embodiments, the specified parental contribution information is recorded in memory and/or other storage.

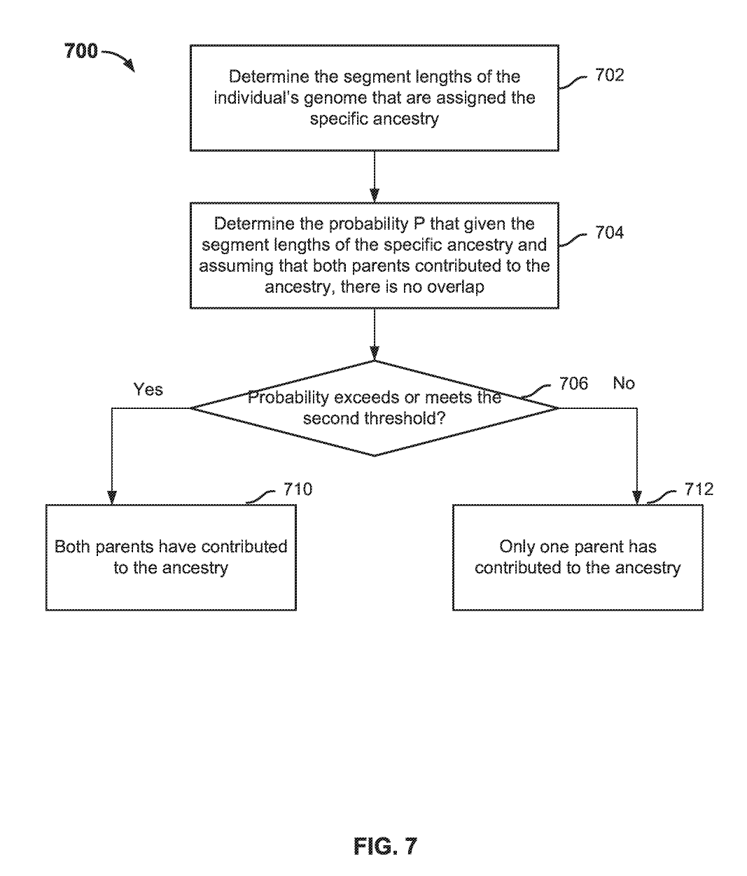

If, however, there is no confirmed region of overlap, further analysis is needed because it is possible that there is only one parent contributing to the specific ancestry, and it is also possible that both parents contributed to the specific ancestry, but their contributions occur on different parts of the individual's genome and therefore do not overlap. At 408, parental contribution to the specific ancestry is statistically determined based at least in part on one or more lengths of one or more chromosome segments deemed to be associated with the specific ancestry. Details of the statistical determination are described below in connection with FIG. 7.

At 410, information pertaining to the parental contribution to the specific ancestry is output. In some embodiments, the information is presented to be displayed to a user. In some embodiments, the user is the individual whose genome is analyzed for parental ancestry estimation. The display permits individuals, who are not necessarily geneticists or ancestry analysis experts, to access and understand their genetic ancestry compositions easily. In some embodiments, the information is sent from an ancestry estimation system (e.g., a server) to a client application such as a browser or a standalone application to be displayed in a user interface.

Process 400 can be repeated for the individual's other ancestry assignments (e.g., French, Italian, etc.) to estimate whether one or both parents contributed to each ancestry.

FIG. 5 is a flowchart illustrating an embodiment of a process for determining whether there is at least one confirmed region of overlapping ancestry assignment associated with a specific ancestry. Process 500 can be used to implement 404 of process 400.

At 502, segments of homologous chromosome pairs with the specific ancestry assignment are compared to identify regions of overlapping ancestry assignment. These regions are to-be-confirmed regions. In some embodiments, the start and end positions of segments with the specific ancestry assignment in each chromosome of a homologous chromosome pair are compared to determine whether there is overlap. Referring to FIG. 3 for an example, for a specific ancestry assignment of Scandinavian, region 306 of FIG. 3 is identified as an overlapping region.

At 504, the amount of overlap for each to-be-confirmed region is determined. The amount of overlap of a region can be measured in genetic distance (e.g., in Centimorgans) or physical distance (e.g., in number of bases). Alternative forms of measurement such as a percentage or a proportional value can be used.

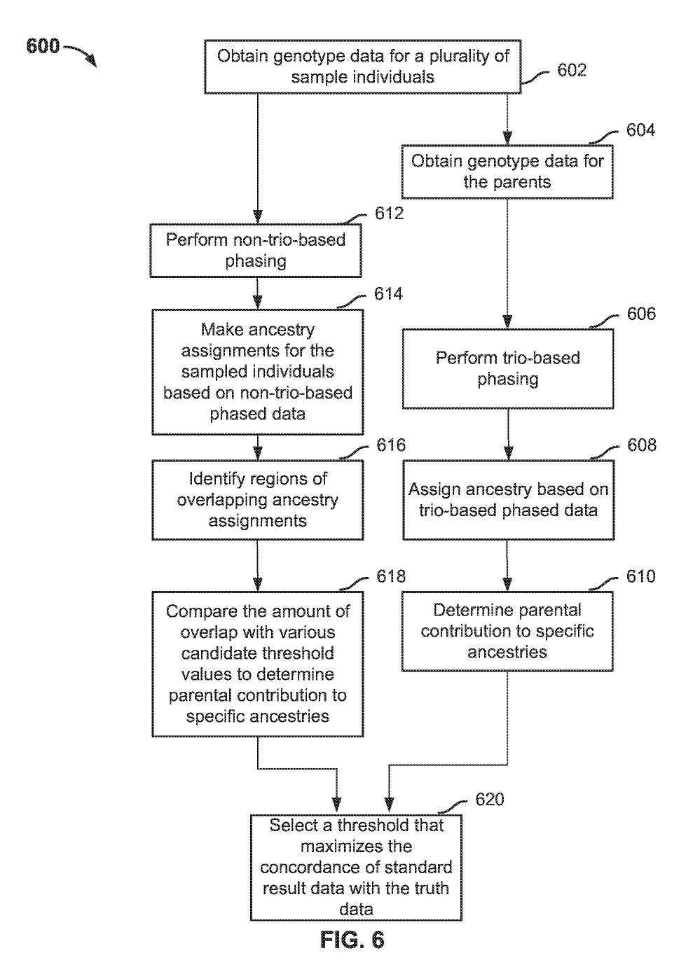

At 506, the amount of overlap of each to-be-confirmed region is compared with a first threshold to determine whether at least one of the to-be-confirmed regions has an overlap amount that at least meets the first threshold. In this example, the first threshold (also referred to as the overlap threshold) is chosen empirically such that an amount of overlap below this threshold is likely due to an error attributed to the assignment technique. For example, a first threshold value of 8 Centimorgans or 8 million bases is used in some embodiments. An example process for determining the first threshold is described below in connection with FIG. 6.

It is determined at 508 whether at least one of the regions has an overlap amount that exceeds or meets the first threshold. If it is determined that there is at least one confirmed region of overlapping ancestry assignment (in other words, it is determined that at least one of the set of the overlapping assignments is deemed to be correctly made,) then both parents have contributed to this ancestry (510), and the flow proceeds to 406 of process 400.

If, however, none of the segments has an overlapping amount that exceeds the first threshold, then it is determined that the segments have no confirmed region of overlapping ancestry assignment (512). One possible cause for no overlap is that there is only one parent contributing to the specific ancestry. Another possible cause is that both parents contribute to the specific ancestry, but their contributions occur on different parts of the individual's genome and do not overlap. Additional statistical analysis (e.g., 408 of process 400) is performed to determine which possibility is more likely and make a determination of parental ancestry contribution accordingly.

Referring to 404 of process 400 and specifically 506 of process 500, FIG. 6 is a flowchart illustrating an embodiment of a process for determining the overlap threshold. A grid search is used in process 600 to determine the overlap threshold (the first threshold used in 506 of process 500). Process 600 can be performed on a system such as 100.

At 602, genotype data for a plurality of sample individuals is obtained. The genotype data is obtained using the same technique as what was used to obtain the individual's genomic data (e.g., using the same type of DNA microarray). In this case, it is assumed that the genotype data for the plurality of sample individuals is also unphased; therefore, if subject to the same phasing process (e.g., a statistical phasing process without using parents' genotype data), the same type of phasing errors mentioned above would result.

At 604, genotype data for the sample individuals' parents is also obtained. In this example, the genotype data for the sample individual's parents is phased. Any appropriate phasing technique can be used to perform the phasing for the parents' data.

At 606, for each sample individual, trio-based phasing is performed using the genotype data of the sample individual as well as the sample individual's parents to produce corresponding phased genotype data for the sample individual. Trio-based phasing is a known phasing technique that uses the parents' genotype data and the individual's unphased genotype readings to determine the individual's phased genotype. For example, suppose that the unphased genotype reading for the sample individual is G/A-T/T-C/A at a particular location, and it is known that Mom has a corresponding chromosome segment of G-T-A and Dad has a corresponding chromosome segment A-T-C at the same location. The individual's unphased reading can then be phased accurately as G-T-A and A-T-C using the trio-based phasing technique. In this example, the phased genotype data also includes information of which parent contributed to which segment (e.g., G-T-A from Mom, A-T-C from Dad, etc.).

At 608, ancestry assignments are made for the plurality of sample individuals based on phased data obtained using trio-based phasing, using the same technique as employed by 402 of process 400, where an ancestry composition tool such as the Geographic Ancestry Analyzer® or the like is used to determine the ancestry assignment information for the sample individuals. The ancestry assignment and the trio-based phased genotype data together specify which chromosome segment and corresponding ancestry assignment is inherited from (or equivalently, contributed by) which parent (e.g., chromosomes 1, position 1-position 15, Scandinavian, inherited from parent 1; chromosome 1, position 16-position 20, German, inherited from parent 2, etc.).

At 610, parental contributions to one or more specific ancestries are determined. In some embodiments, for each sample individual, it is determined whether a specific ancestry is attributed to one parent, two parents, or both. In some embodiments, the determination is made by inspecting the ancestry assignment data obtained in 608 and the trio-based phased genotype data. This set of data is referred to as truth data because it should contain very little phasing error. Table 1 illustrates a set of example truth data. Note that the table only keeps track of ancestry contributions by 1 or 2 parents, and if the individual does not have a particular ancestry, then the corresponding table entry is null. Although a table is shown in this example, other data formats for recording the truth data such as lists, collections, etc. can also be used.

TABLE 1
(Truth Data)

| | German | Italian | Scandinavian | | |
|---|---|---|---|---|---|
| Sample | ancestry | ancestry | ancestry | Chinese | |
| Individual's | inherited | inherited | inherited | ancestry | |
| ID | from | from | from | inherited from | . . . |
| 1 | 1 parent | 2 parents | — | 1 parent | |
| 2 | 2 parents | 1 parent | 1 parent | — | . . . |
| . . . | . . . | . . . | . . . | . . . | . . . |

The truth data will be compared with regular data that is output by the regular phasing process using various overlap thresholds.

At 612, for each sample individual, non-trio-based phasing is performed. The phasing technique that is employed is the same as the phasing technique used to obtain the phased data used by process 400, which does not require the individuals' parents' genotype data and which may result in phasing errors.

At 614, ancestry assignments are made for the plurality of sample individuals based on phased data obtained using non-trio-based phasing, using the same technique as employed by 402 of process 400.

At 616, for the sample individuals, segments of homologous chromosome pairs with the specific ancestry assignment are compared to identify regions of overlapping ancestry assignment in a manner similar to 502 of process 500.

At 618, for specific ancestries for a sample individual, the amount of overlap is compared with various candidate threshold values to determine whether one parent or both parents contributed to the specific ancestries. Specifically, if the amount of overlap at least meets a candidate threshold, then it is determined that two parents contributed to the specific ancestry; otherwise, one parent contributed. The result of the determined parental contribution is referred to as standard result data because it is obtained using the standard process using standard data. Tables 2 and 3 are examples of standard result data for candidate thresholds of 1 and 2. Similar tables can be constructed for other candidate threshold values. The number of candidate threshold values being tested is configurable and depends on implementation; in some embodiments, 20 threshold values are tested.

TABLE 2
(candidate threshold = 1)

| | German | Italian | Scandinavian | | |
|---|---|---|---|---|---|
| Sample | ancestry | ancestry | ancestry | Chinese | |
| Individual's | inherited | inherited | inherited | ancestry | |
| ID | from | from | from | inherited from | . . . |
| 1 | 2 parents | 1 parent | — | 1 parent | . . . |
| 2 | 2 parents | 2 parents | 2 parents | — | . . . |
| . . . | . . . | . . . | . . . | . . . | . . . |

TABLE 3
(candidate threshold = 2)

| | German | Italian | Scandinavian | | |
|---|---|---|---|---|---|
| Sample | ancestry | ancestry | ancestry | Chinese | |
| Individual's | inherited | inherited | inherited | ancestry | |
| ID | from | from | from | inherited from | . . . |
| 1 | 2 parents | 1 parent | — | 1 parent | . . . |
| 2 | 2 parents | 1 parent | 1 parent | — | . . . |
| . . . | . . . | . . . | . . . | . . . | . . . |

At 620, the result from 610 (e.g., truth data of Table 1) and the result from 618 (e.g., standard result data of Table 2, Table 3, etc.) are compared to select a threshold that will lead to maximal concordance of the standard result data with the truth data. In some embodiments, the candidate threshold that yields the highest number of matches with the truth data is selected. Take the examples above, suppose that candidate threshold values of 1 and 2 are tested to yield Tables 2 and 3, because Table 3 has a higher number of matches with Table 1 than Table 2 does, a threshold of 2 is selected as the overlap threshold.

Referring to 408 of process 400, given that there is no confirmed region of overlapping ancestry that corresponds to a specific ancestry, whether the parental contribution to the specific ancestry is made by one parent or both parents is determined statistically. There are a number of ways to make the statistical determination. FIG. 7 is a flowchart illustrating an embodiment of a process to statistically determine whether the parental contribution to the specific ancestry is deemed to be made by one parent or by both parents of the individual. Process 700 can be performed to implement 408 of process 400.

At 702, the segment lengths of the individual's genome that are assigned the specific ancestry are determined. The segment length can be expressed in physical distance or genetic distance, a percentage or proportion, or any other appropriate measurement. For purposes of discussion, assume that there are n such segments, the segment lengths corresponding to segments 1-n are denoted as L₁, L₂, . . . Lₙ.

At 704, the probability, that given the segment lengths of the specific ancestry the individual has and the assumption that both parents contributed to the ancestry, there is no overlap is determined.

In some, the probability P is computed as:

$$P = \frac{( {L}_{t} - {L}_{1} )}{{L}_{t}} \frac{( {L}_{t} - {L}_{1} - {L}_{2} )}{{L}_{t}} … \frac{( {L}_{t} - {L}_{1} - {L}_{2} - … - {L}_{n} )}{{L}_{t}} , \qquad ( 1 )$$

where Lₜ is the total length of the genome on which the segments may reside.

In some embodiments, the probability corresponds to a p-value determined based on a statistical model. As used herein, the p-value refers to the probability of obtaining a test statistic result that is close to what is observed, assuming that the null hypothesis is true. In some embodiments, the null hypothesis is that given the total amount of the individual's genome that is assigned the particular ancestry and both parents contributed to this ancestry (which can be computed by summing the lengths of the chromosome segments assigned to the specific ancestry), there is no overlap. The statistical model is built by observing a sample population of individuals for whom the total amount of overlap for an ancestry assignment and the parental contributions for the ancestry are known. In some embodiments, the statistical model is based on simulations of inheritance rules (e.g., permutations of segments inherited from the parents) and specifies segments that correspond to the specific ancestry and the length of the segments. In such a model, the greater the amount of overlap, the smaller the p-value, indicating that it is less likely that the null hypothesis is true.

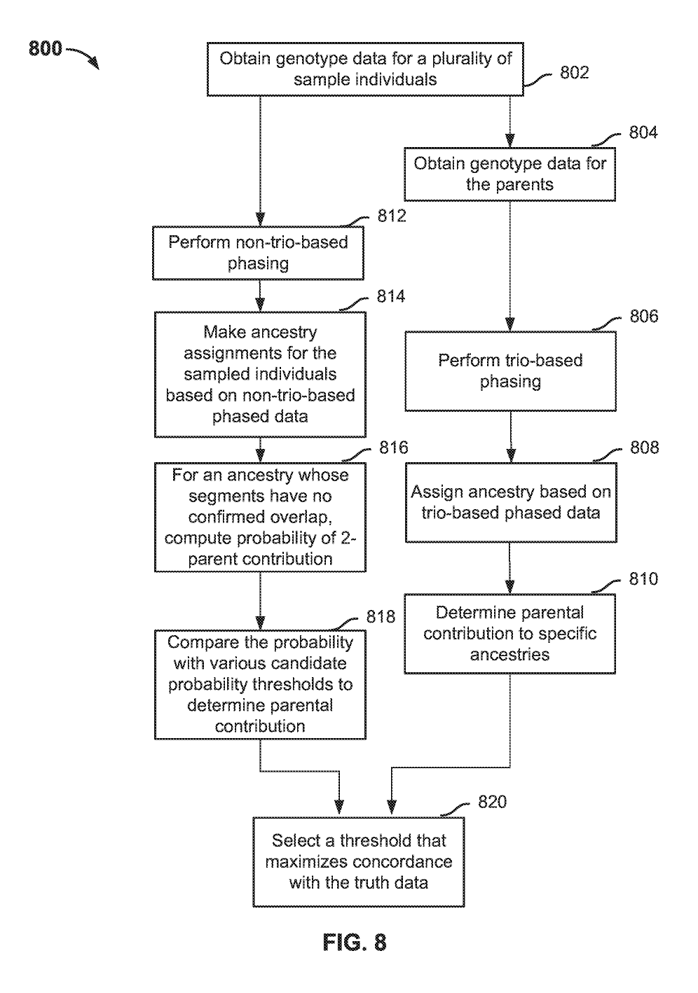

At 706, the determined probability is compared with a second threshold (referred to as the probability threshold). In this example, the second threshold is chosen empirically, such that below the threshold it is deemed unlikely that both parents have contributed to the ancestry but the resulting segments do not overlap. An example process for determining the second threshold is described below in connection with FIG. 8. In some embodiments, the second threshold corresponds to a probability value of 10e-5. At 710, the probability at least meets the threshold, and it is determined that both parents have contributed to the ancestry. At 712, the probability does not at least meet the threshold, and it is determined that only a single parent has contributed to the ancestry.

Referring to 408 of process 400 and specifically 706 of process 700, FIG. 8 is a flowchart illustrating an embodiment of a process for determining the probability threshold. A grid search is used in process 800 to determine the probability threshold (the second threshold used in 706 of process 700). Process 800 can be performed on a system such as 100.

In 802-810, a plurality of sample individuals' and their parents' genotype data is used to generate truth data in a manner similar to 602-610 of process 600. Details of these steps are therefore not repeated.

In 812-818, the plurality of sample individuals' genotype data is phased using a non-trio-based phasing technique and various candidate probability thresholds are used to make parental contribution determinations. 812-814 are similar to 612-618 of process 600; thus, details of these steps are not repeated.

At 816, for the sample individuals, for each ancestry whose corresponding segments have no confirmed region of overlap, the probability of the ancestry being inherited from two parents is computed. In some embodiments, the probability is computed based on the segment lengths and equation (1) as discussed above. In some embodiments, the probability is computed as a p-value as discussed above.

At 818, the probabilities computed in 816 are compared with various candidate probability thresholds to determine whether one parent or both parents contributed to each specific ancestry. Specifically, if the computed probability at least meets a candidate threshold, then it is determined that two parents contributed to the specific ancestry; otherwise, one parent contributed. Tables 4 and 5 are examples of standard result data for candidate thresholds of 5% and 10%. Similar tables can be constructed for other candidate threshold values. The number of candidate threshold values being tested and the actual values used are configurable and depend on implementation.

TABLE 4
(candidate threshold = 5%)

| | German | Italian | Scandinavian | | |
|---|---|---|---|---|---|
| Sample | ancestry | ancestry | ancestry | Chinese | |
| Individual's | inherited | inherited | inherited | ancestry | |
| ID | from | from | from | inherited from | . . . |
| 1 | 2 parents | 1 parent | — | 1 parent | . . . |
| 2 | 2 parents | 2 parents | 2 parents | — | . . . |
| . . . | . . . | . . . | . . . | . . . | . . . |

TABLE 5
(candidate threshold = 10%)

| | German | Italian | Scandinavian | | |
|---|---|---|---|---|---|
| Sample | ancestry | ancestry | ancestry | Chinese | |
| Individual's | inherited | inherited | inherited | ancestry | |
| ID | from | from | from | inherited from | . . . |
| 1 | 2 parents | 1 parent | — | 1 parent | . . . |
| 2 | 2 parents | 1 parent | 1 parent | — | . . . |
| . . . | . . . | . . . | . . . | . . . | . . . |

At 820, the result from 810 (e.g., Table 1) and the result from 818 (e.g., Table 4, Table 5, etc.) are compared to select a threshold that will lead to maximal concordance with the truth data. In some embodiments, the candidate threshold that yields the highest number of matches with the truth data is selected. Take the examples above, suppose that only candidate threshold values of 5% and 10% are tested to yield Tables 4 and 5, because Table 5 has a higher number of matches with Table 1 than Table 4 does, a threshold of 10% is selected as the probability threshold.

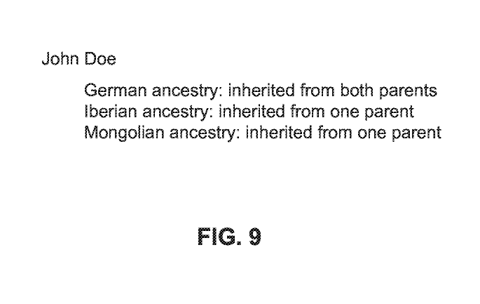

Referring to 410 of process 400, the estimated parental contributions are presented to the user to be displayed. FIG. 9 is an embodiment of a user interface screen illustrating the estimated parental ancestry contributions. In this example, different ancestries are listed, and parental contributions to each ancestry are determined using the technique described above and shown. The individual, John Doe, is the user who initiated the parental contribution estimation process. Allowing the user to estimate and review his own parental ancestry contributions improves the user's comprehension of his/her ancestry composition and genetic data.

Other user interfaces displaying the information in different formats can be used (e.g., displaying different ancestries using different colors, patterns, shades, etc.). For example, the display can further incorporate parent information. For example, if the genotype data of at least one of the parents of the individual is also available in the database or from another source, the determination of whether one parent or both parents contributed to a particular ancestry can be made by examining what ancestries are carried on which of set of phased chromosomes, one set inherited from the father and one set inherited from the mother.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method comprising:
obtaining a set of ancestry assignment data associated with an individual's genotype data, at least some of the ancestry assignment data indicating that one or more segments of the individual's genotype data are deemed to be associated with a specific ancestry;
determining whether in the individual's genotype data there is at least one confirmed region of overlapping ancestry assignment associated with the specific ancestry;
in the event that it is determined that there is at least one confirmed region of overlapping ancestry assignment associated with the specific ancestry:
specifying that parental contribution of the specific ancestry is made by both parents of the individual;
in the event that it is determined that there is no confirmed region of overlapping ancestry assignment associated with the specific ancestry:
statistically determining whether the parental contribution to the specific ancestry is made by only one parent of the individual or by both parents of the individual, the determination being based at least in part on one or more lengths of the one or more segments deemed to be associated with the specific ancestry; and
outputting information pertaining to the parental contribution to the specific ancestry.

2. The method of claim 1, wherein the determining of whether in the individual's genotype data there is at least one confirmed region of overlapping ancestry assignment associated with the specific ancestry is based at least in part on an amount of overlap in a to-be-confirmed region of overlapping ancestry assignment.

3. The method of claim 1, wherein the determining of whether in the individual's genotype data there is at least one confirmed region of overlapping ancestry assignment associated with the specific ancestry includes:
identifying one or more to-be-confirmed regions of overlapping ancestry assignment in the individual's homologous chromosome pairs;
determining an amount of overlap in a to-be-confirmed region;
comparing the amount of overlap with a threshold; and
in the event that the amount of overlap at least meets the threshold, determining that there is at least one confirmed region of overlapping ancestry assignment associated with the specific ancestry.

4. The method of claim 3, wherein the threshold is determined based at least in part on a grid search based at least in part on genotype data of a plurality of sample individuals and genotype data of parents of the plurality of sample individuals.

5. The method of claim 4, wherein the grid search includes:
generating truth data of the plurality of sample individual's parental contributions to ancestries based at least in part on the genotype data of the plurality of sample individuals and the genotype data of the parents of the plurality of sample individuals;
generating standard result data of the plurality of sample individual's parental contributions to ancestries based at least on the genotype data of the plurality of sample individuals but not the genotype data of the parents of the plurality of sample individuals, and based at least in part on a plurality of candidate thresholds; and
selecting a threshold that maximizes concordance of the standard result data with the truth data.

6. The method of claim 3, wherein the threshold is measured in genetic distance or physical distance.

7. The method of claim 3, wherein the determining whether in the individual's genotype data there is at least one confirmed region of overlapping ancestry assignment associated with the specific ancestry further includes:
determining one or more amounts of overlap for one or more other to-be-confirmed regions and comparing the one or more amounts of overlap for one or more other to-be-confirmed regions with the threshold; and
in the event that no amount of overlap at least meets the threshold, determining that there is no confirmed region of overlapping ancestry assignment associated with the specific ancestry.

8. The method of claim 1, wherein the statistically determining whether the parental contribution to the specific ancestry is made by only one parent of the individual or by both parents of the individual includes:
determining a probability that given the one or more segments deemed to be associated with the specific ancestry and an assumption that both parents of the individual contributed to the specific ancestry, there is no confirmed region of overlapping ancestry assignment associated with the specific ancestry;
in the event that the probability at least meets a threshold, specifying that the parental contribution of the specific ancestry is made by both parents of the individual; and
in the event that the probability does not at least meet the threshold, specifying that the parental contribution of the specific ancestry is made by only one parent of the individual.

9. The method of claim 8, wherein the threshold is determined based at least in part on a grid search based at least in part on genotype data of a plurality of sample individuals and parents of the plurality of sample individuals.

10. The method of claim 9, wherein the grid search includes:
generating truth data of the plurality of sample individual's parental contributions to ancestries based at least in part on the genotype data of the plurality of sample individuals and genotype data of the parents of the plurality of sample individuals;
generating standard result data of the plurality of sample individual's parental contributions to ancestries based at least on the genotype data of the plurality of sample individuals but not the genotype data of the parents of the plurality of sample individuals, and based at least in part on a plurality of candidate thresholds; and
selecting a threshold that maximizes concordance of the standard result data with the truth data.

11. The method of claim 1, wherein the information pertaining to the parental contribution to the specific ancestry is displayed to the individual.

12. A system, comprising:
one or more processors configured to:
obtain a set of ancestry assignment data associated with an individual's genotype data, at least some of the ancestry assignment data indicating that one or more segments of the individual's genotype data are deemed to be associated with a specific ancestry;
determine whether in the individual's genotype data there is at least one confirmed region of overlapping ancestry assignment associated with the specific ancestry;
in the event that it is determined that there is at least one confirmed region of overlapping ancestry assignment associated with the specific ancestry, specify that parental contribution of the specific ancestry is made by both parents of the individual;
in the event that it is determined that there is no confirmed region of overlapping ancestry assignment associated with the specific ancestry, statistically determine whether the parental contribution to the specific ancestry is made by only one parent of the individual or by both parents of the individual, the determination being based at least in part on one or more lengths of the one or more segments deemed to be associated with the specific ancestry; and
output information pertaining to the parental contribution to the specific ancestry; and
one or more memories coupled to the one or more processors and configured to provide the one or more processors with instructions.

13. The system of claim 12, wherein to determine whether in the individual's genotype data there is at least one confirmed region of overlapping ancestry assignment associated with the specific ancestry is based at least in part on an amount of overlap in a to-be-confirmed region of overlapping ancestry assignment.

14. The system of claim 12, wherein to determine whether in the individual's genotype data there is at least one confirmed region of overlapping ancestry assignment associated with the specific ancestry includes to:
identify one or more to-be-confirmed regions of overlapping ancestry assignment in the individual's homologous chromosome pairs;
determine an amount of overlap in a to-be-confirmed region;
compare the amount of overlap with a threshold; and
in the event that the amount of overlap at least meets the threshold, determine that there is at least one confirmed region of overlapping ancestry assignment associated with the specific ancestry.

15. The system of claim 14, wherein the threshold is determined based at least in part on a grid search based at least in part on genotype data of a plurality of sample individuals and genotype data of parents of the plurality of sample individuals.

16. The system of claim 15, wherein the grid search includes to:
generate truth data of the plurality of sample individual's parental contributions to ancestries based at least in part on the genotype data of the plurality of sample individuals and the genotype data of the parents of the plurality of sample individuals;
generate standard result data of the plurality of sample individual's parental contributions to ancestries based at least on the genotype data of the plurality of sample individuals but not the genotype data of the parents of the plurality of sample individuals, and based at least in part on a plurality of candidate thresholds; and
select a threshold that maximizes concordance of the standard result data with the truth data.

17. The system of claim 14, wherein the threshold is measured in genetic distance or physical distance.

18. The system of claim 14, wherein to determine whether in the individual's genotype data there is at least one confirmed region of overlapping ancestry assignment associated with the specific ancestry further includes to:
determine one or more amounts of overlap for one or more other to-be-confirmed regions and compare the one or more amounts of overlap for one or more other to-be-confirmed regions with the threshold; and
in the event that no amount of overlap at least meets the threshold, determine that there is no confirmed region of overlapping ancestry assignment associated with the specific ancestry.

19. The system of claim 12, wherein to statistically determine whether the parental contribution to the specific ancestry is made by only one parent of the individual or by both parents of the individual includes to:
determine a probability that given the one or more segments deemed to be associated with the specific ancestry and an assumption that both parents of the individual contributed to the specific ancestry, there is no confirmed region of overlapping ancestry assignment associated with the specific ancestry;
in the event that the probability at least meets a threshold, specify that the parental contribution of the specific ancestry is made by both parents of the individual; and
in the event that the probability does not at least meet the threshold, specify that the parental contribution of the specific ancestry is made by only one parent of the individual.

20. A computer program product, the computer program product being embodied in a tangible computer readable storage medium and comprising computer instructions for:
obtaining a set of ancestry assignment data associated with an individual's genotype data, at least some of the ancestry assignment data indicating that one or more segments of the individual's genotype data are deemed to be associated with a specific ancestry;
determining whether in the individual's genotype data there is at least one confirmed region of overlapping ancestry assignment associated with the specific ancestry;
in the event that it is determined that there is at least one confirmed region of overlapping ancestry assignment associated with the specific ancestry:
specifying that parental contribution of the specific ancestry is made by both parents of the individual;
in the event that it is determined that there is no confirmed region of overlapping ancestry assignment associated with the specific ancestry:
statistically determining whether the parental contribution to the specific ancestry is made by only one parent of the individual or by both parents of the individual, the determination being based at least in part on one or more lengths of the one or more segments deemed to be associated with the specific ancestry; and
outputting information pertaining to the parental contribution to the specific ancestry.
